(19) United States
(12) Patent Application Publication
(10) Pub. No.: US 2026/0311339 A1
(43) Pub. Date: Oct. 8, 2026

(54) MULTIPLE LOSS FUNCTIONS FOR ROBUST SEGMENTATION OF MULTIPLE OBJECTS

(71) Applicant: IB Lab GmbH, Wien (AT)

(72) Inventor: Giacomo DEODATO, Wien (AT)

(21) Appl. No.: 19/490,296

(22) PCT Filed: Jun. 5, 2023
(86) PCT No.: PCT/EP2023/064976

§ 371 Date: Dec. 4, 2025

(51) Int. Cl. G06T 7/12; G06N 20/00; G16H 30/40

(52) U.S. Cl. CPC G06T 7/12; G06N 20/00; G16H 30/40

(57) ABSTRACT

A computer-implemented method for training a machine learning model (15), wherein the machine learning model (15) is configured to use a medical image (25) as an input for generating an output comprising at least two image segments (27, 28) of the medical image (25), wherein the training comprises optimizing a loss function measuring the predictive performance of the machine learning model (15) for a set of training data to obtain a set of optimal model parameters of the machine learning model (15) as result of the optimization, wherein the loss function comprises a loss contribution that depends on a geometrical relationship between at least two of the image segments (27, 28).

The invention concerns a computer-implemented method for training a machine learning model, wherein the machine learning model is configured to use a medical image as an input for generating an output comprising at least two image segments of the medical image, wherein the training comprises optimizing a loss function measuring the predictive performance of the machine learning model for a set of training data to obtain a set of optimal model parameters of the machine learning model as result of the optimization. The invention also concerns a method for segmentation of anatomical structures in a medical image comprising the steps of: training a machine learning model to obtain a set of optimal model parameters according to the training method mentioned above, and segmenting anatomical structures in said medical image by using said machine learning model with said optimal model parameters. That method may be a method for multi-label segmentation of anatomical structures in a medical image. The scope of the invention also extends to a set of optimal model parameters for a machine learning model, wherein the machine learning model is configured to use a medical image as an input for generating an output comprising at least two image segments of the medical image. The scope of the invention also extends to a computer program for segmentation, in particular multi-label segmentation, of anatomical structures in a medical image, wherein the computer program comprises the set of optimal model parameters and the machine learning model according to the invention and instructions to cause a data processing apparatus to execute the step of segmenting anatomical structures in said medical image by using said machine learning model with said optimal model parameters. Finally, the invention also extends to a computer-readable medium having stored thereon that computer program.

Accurate vertebral segmentation and labeling is of fundamental importance to automate spine related clinical tasks including individual vertebral pathologies (e.g. vertebral fracture detection, tumor detection), spinal deformities (e.g. scoliosis), clinically significant measurements (e.g. vertebral rotation, vertebral height, Gennant score), and surgical applications (e.g. planning and postoperative assessment).

General spine assessment presents a number of challenges. Spine morphology is heterogeneous, it depends on the patient's age as well as vertebral and spinal pathologies including different numbers of vertebrae. Moreover, spine assessment may be conducted using different modalities depending on the task to be performed. The main modalities are Computed Tomography (CT), Magnetic Resonance Imaging (MRI), X-Ray and Dual-Energy X-ray Absorptiometry (DEXA). The different modalities highlight different components of the spine anatomy, resulting in different data formats (2D and 3D) and imaging quality. Finally, the automation of these tasks is influenced by other factors such as different spinal views (frontal, transversal or lateral), variable field-of-view, foreign objects presence and other imaging artifacts.

Most of the prior art work on spine images for vertebral detection, segmentation and labeling has been done on MRI and CT, with a smaller focus on X-Ray. Nonetheless, the methodologies applied on a specific imaging modality can generally be applied to the others by providing new training data and adapting the model to work with a different format (e.g. from 2D to 3D).

Vertebral detection and segmentation algorithms generally suffer from three main issues: false detection, aggregate detection and impossible detection. False detection happens when a vertebra is detected in a region of the image where no vertebra is present, outside of the spine. Aggregate detection happens when, due to vertebral pathologies or poor image quality, two or more vertebrae are detected and/or segmented together. While instance segmentation algorithms may perform better than semantic segmentation ones, they are still exposed to the aforementioned issues.

The main approaches available in the literature compute the vertebral labels in three different ways: regression, classification and segmentation.

Regression and classification approaches are heavily dependent on the vertebral detection algorithm. Given a vertebral patch, obtained by a bounding box or a segmentation mask, they try to predict the corresponding vertebral label using a regression or multi-label classification approach.

Moreover, since vertebrae belonging to the same segment look similar to each other, predictions provided by the classification or regression model are usually not enough to provide a correct vertebral labeling because they may label different vertebrae belonging to the same segment in the same way. For this reason these labels are usually post-processed together with the corresponding vertebral position to obtain a coherent labeling scheme where each vertebra has a unique label and consecutive labels are assigned to adjacent vertebrae.

On the other hand, labeling by segmentation approaches using the semantic segmentation paradigm, use models with a predefined number of output channels where each channel corresponds to a vertebra label. While this approach may take advantage of the information encoded in the whole image (because no patches are extracted) it still suffers from some of the limitations of the previous approaches because similar vertebrae may have the same or inverted labels. Finally, the main limitation of this approach is the fixed number of outputs (usually 24, 7 cervical+12 thoracic+5 lumbar) which does not accurately reflect the variable spine length seen in reality due to variable numbers of thoracic and/or lumbar vertebrae (the latter in 10% of the people) or cropped spine images.

CN114693719A shows a spine image segmentation method based on a model architecture named “3D-SE-V-net”. The main innovation is the use of Squeeze and Excite units inside the V-Net architecture. The loss function is the standard cross-entropy. The model has five outputs (background, disc, disc abnormality, spinal canal, articular process). The outputs are evaluated separately, i.e. using separate, individual loss functions, and not connected.

CN114140480A shows a method for semantic segmentation of thermal infrared electrical equipment images based on edge-assisted learning, wherein a cross-entropy loss function and a binary cross-entropy loss function are combined. The main innovation is the use of an edge prediction branch. The loss functions are standard cross-entropy. The model has two outputs: one for semantic segmentation and another one for edge segmentation. The outputs are not connected, they are jointly learned by combining the corresponding two losses, which are evaluated separately for each respective output.

CN109785336A shows an image segmentation method based on multi-path convolutional neural network model, wherein the feature image output by the first layer of the auxiliary convolution layer of the auxiliary path is used as the third feature image. The main innovation is the use of auxiliary paths in the model. The loss function is standard cross-entropy. The model has only one output: the segmentation mask.

CN114305473A and CN113240698A show a body composition automatic measurement system, wherein the loss function trained by the segmentation module has multiple terms. For a 2D abdominal CT image, in the medical image segmentation task, the multi-class cross-entropy loss function and the Dice loss function are combined to obtain four types of skeletal muscles. The main innovation is the use of the whole CT volume to segment skeletal muscles around L3. The loss function is a combination of cross-entropy and dice loss. The model has one output mask with five classes.

The invention is based on the realization that using individual patches or masks separately does not allow the overall algorithm to exploit prior knowledge about the problem. Generally, the human body always follows a certain anatomy that can be translated into prior knowledge, e.g. of the bone structure.

For example regarding the spine only for purposes of illustrating the above general concepts and without limiting the scope of the invention, this prior knowledge can be encoded in the following rules:
a. Vertebrae are stacked on top of each other following the spine curve;
b. Vertebrae that are close to each other have a similar morphology;
c. Spine segments contain adjacent vertebrae;
d. Derived from b and c: spine segments can be labeled according to the vertebral morphology;
e. Spine segments can be located and labeled according to their position relative to other key anatomical elements (e.g. ribs for the thoracic segment, pelvis for the lumbar segment, head and shoulders for the cervical segment);
f. Spine segments always follow the same order, cranial to caudal: cervical, thoracic, lumbar and sacral segments.

In this example, that prior knowledge allows to derive a set of particular topical constraints for vertebral labeling and segmentation:
1. Vertebrae belonging to the same segments are close to each other in the spine;
2. Vertebrae that are closer to each other have a similar morphology;
3. Vertebrae belonging to different segments cannot alternate in the spine;
4. The different spine segments always follow the same order: cervical, thoracic, lumbar and sacral spine, from top to bottom;
5. Vertebral segments can be labeled according to the vertebral shape;
6. Vertebral segments, and corresponding individual vertebrae, can be labeled according their position relative to other key anatomical elements present in the image (e.g. ribs for the thoracic segment, pelvis for the lumbar segment, head and shoulders for the cervical segment)

It is an object of the invention to propose a method and related implementations as defined at the outset, that overcomes or at least improves on the challenges mentioned above in relation to the prior art of segmenting medical images, and that takes advantage of prior knowledge of the human body.

As mentioned at the outset, the invention has multiple aspects that are interrelated and each separately are embodiments of the invention. Those aspects are therefore discussed individually in the following and are claimed independently to cover the full scope of the invention. Generally, the invention is embodied in a specific method for training a machine learning model as well as the resulting trained model represented by a set of model parameters, the use of that trained model, such as a method involving the trained model to perform medical image segmentation, and finally the computer program implementing the above methods as well as a computer-readable storage medium having stored thereon such a computer program.

A computer-implemented method for training a machine learning model as defined in the outset, wherein the loss function comprises a loss contribution that depends on a geometrical relationship between at least two of the image segments.

When the term “computer-implemented method” is used in the present disclosure, it refers to a method being performed on one or more computers, on a system implemented by one or more computers, in a computer network connecting two or more computers, or generally on a programmable apparatus or system, wherein at least one step of the method is performed by means of a computer.

A machine learning model, as used in this patent application, comprises algorithms and data structures, which are adapted or adaptable to a given input and corresponding output. The process of this adaptation is called training. During this process, the machine learning model is adapted to generate the given output or at least an output similar to the given output, when provided with the given input. The given input and given output as well as the measure of similarity are also part of the training and influence the trained machine learning model. The given input and given output are also called the training data. They may be separate data structures (for example, when the outputs have a different data type than the inputs, such as images and labels respectively) or they may correspond to parts of the same data structure (for example, when outputs and inputs of the same data type, such as text). Those parts and properties of the machine learning model that do not change during training are called the model architecture. There is a wide variety of possible architectures presently available, ranging from linear regression to deep neural networks. The choice of architecture directly impacts not only the required training data but also the costs and performance of the training as well as that of the trained model and the quality of its predictions, i.e. of the generated outputs for a new input that has not been part of the training data.

One exemplary class of architectures that is suitable for the machine learning model employed in the present disclosure is that of convolutional neural networks. One exemplary choice from that class is a U-net architecture. For example, the machine learning model may comprise at least one convolutional neural network and the optimal model parameters—i.e., those determined by the training—include optimal fixed weights for the at least one convolutional neural network. The training data comprises at least training inputs (the given inputs mentioned above) and training outputs (also called “ground truth”; the given outputs mentioned above). The similarity between the generated outputs and the given outputs is measured by a loss function. The loss function has multiple contributions. For image segmentation—the application discussed in the present disclosure—one contribution relates the generated segmentation to the corresponding expected segmentation according to the ground truth. This contribution, called the “main loss”, typically depends on the overlap between the model outputs and the corresponding ground truth. The present invention proposes another, typically additional loss contribution, called “auxiliary loss”, that depends on a geometrical relationship between at least two of the image segments. The “image segments” are part of the output of the machine learning model. This means that this other additional contribution generally varies with any changes in the geometry of any of the image segments involved in the relationship. The machine learning model may comprise other layers/operations in addition to one or more neural networks (in this example). A loss function can in principle be defined at any level of the model.

The methods of the present invention shall apply to two-dimensional data (projections or slices) as well as three-dimensional data (volumes), wherein both are referred to as “images”. In other words, the term “image” in the present patent application is used in the mathematical sense and means both, two-dimensional and three-dimensional images.

The image segments are generally regions (not necessarily contiguous) of the medical image or partial selections from the medical image, wherein each partial selection delimits one or more areas of the medical image, each being smaller than the total area of the medical image. In other words, image segments refer to generally non-continuous regions, parts, or sections from or within the image. In this disclosure the image segments collectively may cover only a part of the entire image. It is generally not necessary that they collectively cover the entire image. The image segments may each be associated with a segment label indicating a descriptor of the segment, e.g. with the type of an object or group of objects represented in the image segment.

The machine learning model may be configured to generate the at least two image segments as image masks, in particular binary masks, applicable to the medical image. an image mask may be a two-dimensional array or matrix of pixels, for example with the same pixel dimensions as the original image. Each pixel corresponds to a particular location in the original image. In the case of a binary mask, each pixel has a value of 1 or 0. Generally, continuous masks may also be used, with pixel values between 0 and 1, to indicate uncertainty within the segmentation. In case of three-dimensional images, the image mask may be a three-dimensional array of voxels which are used and behave similarly to the pixels described above. Each image mask may be associated with a label for an image segment corresponding to the respective image mask. By applying such an image mask to the medical image, the image content (or objects) designated by the associated label can be selected in the medical image and other parts of the medical image may be removed or filtered.

The training data can comprise a plurality of medical training images and a corresponding plurality of image training segments, wherein each medical training image comprises a graphical representation of parts of an anatomy (e.g. of a human skeleton), wherein the corresponding image training segments indicate the anatomic parts (e.g. the bones) represented in the respective medical training image. This training dataset allows to train the machine learning model for segmenting medical images of a similar anatomy according to the anatomic parts visible in the medical image. In particular, the present invention can be applied for segmenting radiology medical images showing specific parts of the human skeleton into the bones belonging to a specific part, optionally together with labeling those bones according to a predefined medical naming convention, which can be derived during training from the training data.

According to one embodiment of the present invention, the loss contribution may depend on an overlap between at least two of the image segments. From the at least two image segments generated by the machine learning model, their overlap or multiple overlaps (like the images, two-dimensional or three-dimensional) can be determined, and a loss contribution computed, for example proportional, or for example linearly proportional, to the size (area or volume) of the overlap or the total size (total area total volume) of the overlaps. This loss contribution penalizes overlapping segments/masks. The machine learning model is thereby trained to provide an image segmentation without overlapping image segments.

According to another embodiment of the present invention, the geometrical relationship on which the loss contribution depends, may represent a mereology relationship between at least two of the image segments, wherein from the at least two of the image segments a first image segment represents an anatomic whole and at least one second image segment represents an anatomic part of the anatomic whole. In this case, an overlap of the image segments is expected. More specifically, the second image segment is expected to be completely contained within the first image segment.

In this context, the loss contribution may for example depend on an exceedance of the first image segment by the at least one second image segment. From the at least two image segments generated by the machine learning model, a part of the second image segment that exceeds (non-overlaps) the first image segment is determined. A loss contribution can then be computed, for example proportional, for example linearly proportional, to the size (area or volume) of the exceeding part(s). Such a loss contribution penalizes exceedance and thereby trains the machine learning model to generate first and second image segments wherein the first image segment is large enough and positioned to contain the second image segment or the second image segment is small enough and positioned to be contained by the first image segment.

Further in the same context, the loss contribution may depend on the difference between the first image segment and the at least one second image segment. From the at least two image segments generated by the machine learning model, a free space within the first image segment and outside the second image segment can be determined. This free space corresponds to a distance between the different anatomic parts of the anatomic whole.

More specifically, the loss contribution may be proportional, for example linearly proportional, to the size (area of volume) of the difference between the first image segment and the at least one second image segment. This size is the size of the free space mentioned above. In case of multiple anatomic parts belonging to the same anatomic whole, which will be the common case, the size of the difference can be determined between the first image segment and the combination (e.g. a logical disjunction in case of binary masks) of the two or more second image segments. With a loss contribution that is proportional to the size, the machine learning model will be trained to fill the first image segment with the one or more second image segments, i.e. preferring a size of the free space of zero.

According to yet another embodiment, the method involves at least two machine learning models, wherein each machine learning model is separately trained using a separate loss function that comprises a loss contribution that depends on a geometrical relationship representing a mereology relationship between at least two of the output image segments, wherein the machine learning models together generate an output comprising at least three image segments, wherein each machine learning model is trained on a different level of a hierarchy of mereology relationships between the output image segments. Each machine learning model may be trained on its own (for example in parallel) using different loss functions and different parts of the training dataset. Such a hierarchy of mereology relationships could be, for example, with regard to a medical image of a spine, a first image segment selecting the spine as a whole, multiple second image segments selecting different segments of the spine, and multiple third image segments selecting the individual vertebrae of each of the different spine segments. In this case, all second image segments must be contained within the first image segment and each of the third image segments must be contained within one of the second image segments. In general, this type of hierarchy is not limited to single entities at the top level; there may be multiple first image segments as well.

The preceding paragraphs discussed different embodiments corresponding to different particular types of loss contributions for contributing to the “auxiliary loss” defined above. Within the scope of the invention, different such loss contributions can be combined to together form the auxiliary loss. The actual loss function can then be determined by combining, for example adding, the main loss and the auxiliary loss.

Regarding the training dataset, from the image training segments corresponding to one medical training image a first image training segment can for example represent an anatomic whole and at least one second image training segment represent an anatomic part of the anatomic whole, wherein the loss contribution may further depend on the difference between a first image training segment and the at least one second image training segment. The loss contribution may for example be determined by comparing the determined difference with an expected difference. The expected difference may correspond to an expected spacing within the composition between components. Again, in these examples, the determined difference and the expected difference may be the sizes (areas or volumes) of the corresponding non-overlapping parts of the image segments in question. The related loss contribution can then be proportional to the relative difference between the determined difference and the expected difference.

For example, the present invention concerns the use of the method for segmentation of anatomical structures in a medical image as defined in the outset for segmenting a medical image of a spine, a hand, a foot, a cell or an embryo. The set of training data may therefore comprise medical images of the respective class of bodies or body parts.

For the set of optimal model parameters mentioned at the outset for a machine learning model, wherein the machine learning model is configured to use a medical image as an input for generating an output comprising at least two image segments of the medical image, it is foreseen that the set of optimal model parameters has been obtained according to one of the variations of the method defined above.

Referring now to an exemplary embodiment of the invention described in detail along with the drawings, wherein the figures are for purposes of illustrating the present invention and not for purposes of limiting the same:

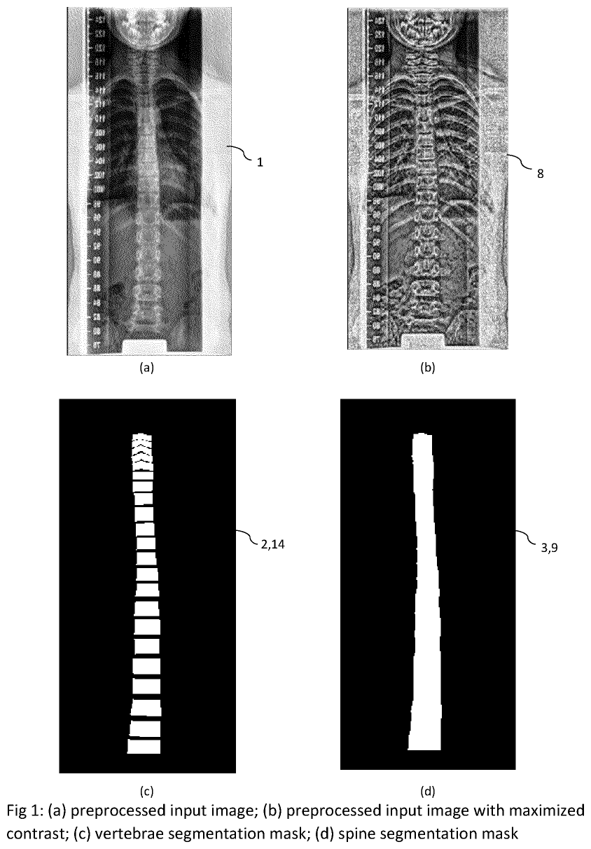

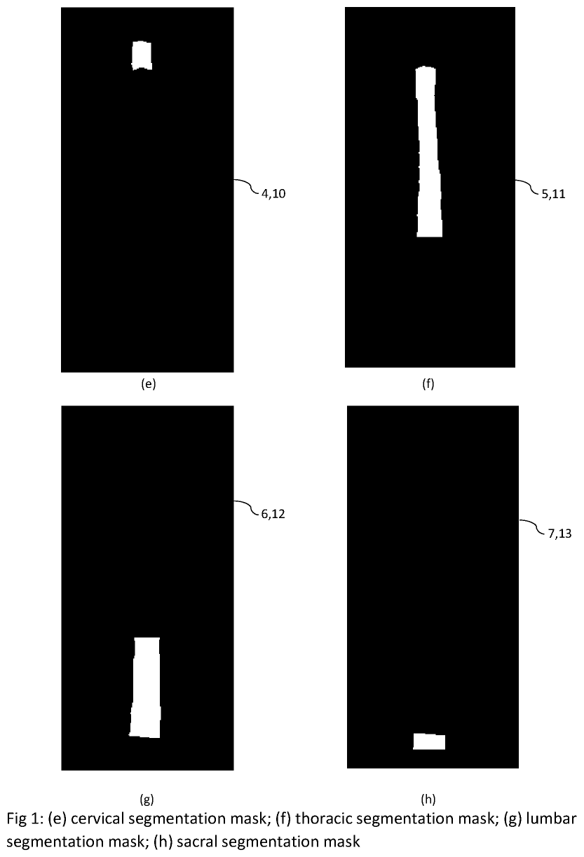

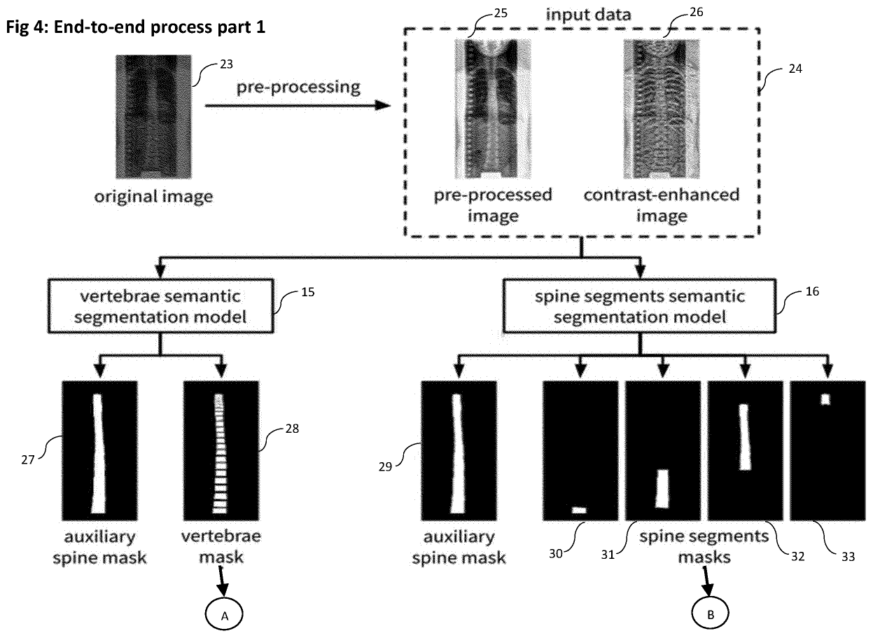

FIG. 1a shows an exemplary pre-processed medical image as it is obtained after pre-processing an original image of an X-ray (see also FIG. 4);

FIG. 1b shows a contrast-enhanced medical image that is obtained from the pre-processed medical image shown in FIG. 1a;

FIGS. 1c and 1d show binary masks for vertebrae segmentation and spine segmentation respectively of the medical image shown in FIG. 1b;

FIG. 1e-h show binary masks for different spine segment segmentation, namely cervical (FIG. 1e), thoracic (FIG. 1f), lumbar (FIG. 1g), and sacral (FIG. 1h) spine segments;

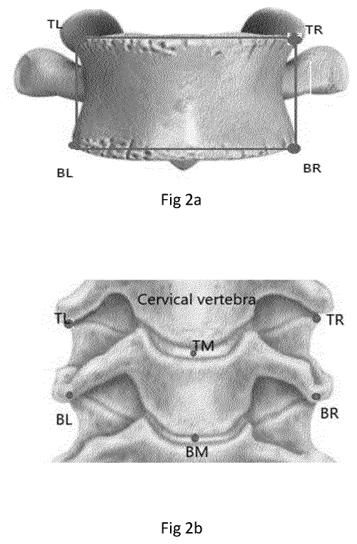

FIGS. 2a and 2b illustrate the labelling of the vertebrae of a human spine for obtaining their output data of the training dataset;

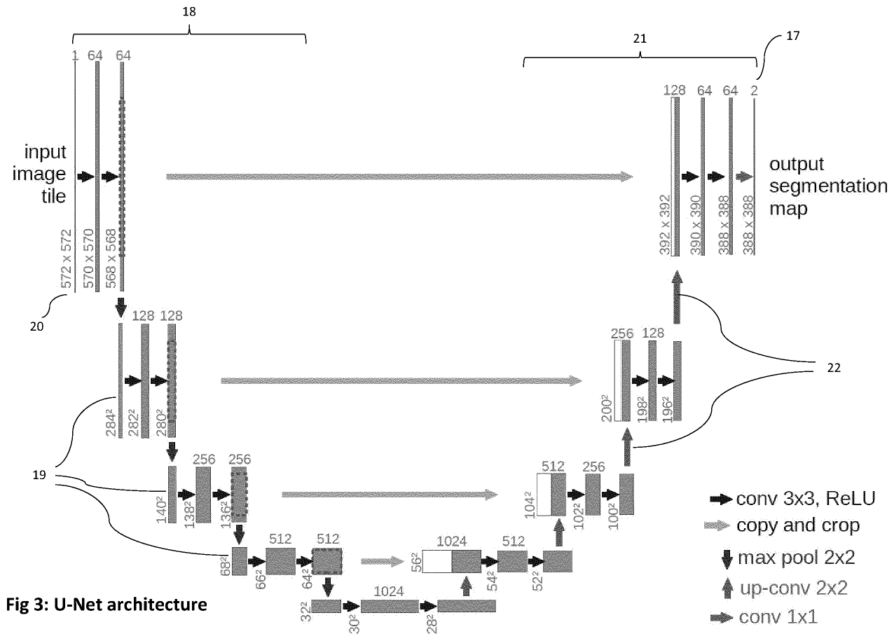

FIG. 3 illustrates the architecture of the machine learning model of the present exemplary embodiment, showing the data structures and operations operating on them when propagating an input medical image through the machine learning model;

FIG. 4 illustrates the processing chain from an original image obtained of an X-ray to the image segments output by two machine learning models, one for vertebrae segmentation and one for spine segment segmentation; and

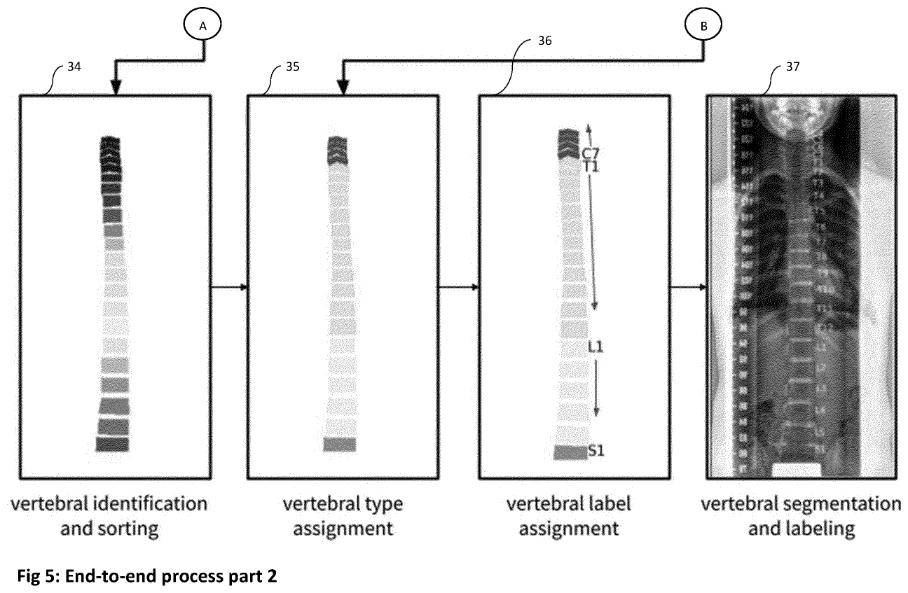

FIG. 5 illustrates the combination of the obtained image segments to obtain vertebral labels, which are finally overlaid with the pre-processed medical image.

Training data can be divided into input data and output data. The input data comprises medical images and the output data comprises image segments of the medical images. For each medical image input data, at least two image segments of the output data are associated. FIG. 1 shows one instance of a combination of a medical image 1 and the associated image segments 2-7. In the present exemplary embodiment, which concerns training a machine learning model for spine image segmentation, input data consists of spine images (specifically: spine X-Rays) and the output data is made of binary masks (or semantic segmentation masks) for the entire spine 3, the individual vertebrae 2 and one for each of the spine segments 4-7 (see FIG. 1).

The original medical image obtained from an imaging device (e.g. an X-ray system) is processed before being fed to the machine learning model in order to generate two input medical images from it: the first medical image 1 corresponds to the normalized (between 0 and 1) image after clipping the pixel values to the 0.2 and 99.8 percentiles of their distribution and resizing the shape so that the aspect ratio remains the same but the smallest dimension between height and width becomes 256; the second medical image 8 is the same as the first medical image 1 after processing the image to maximize the contrast using Contrast Limited Adaptive Histogram Equalization (CLAHE, see Pizer, Stephen M., et al. “Adaptive histogram equalization and its variations.” Computer vision, graphics, and image processing 39.3 (1987): 355-368.). An example of input medical images 1, 8 and corresponding output image segments 2-7 is illustrated in FIG. 1.

The image segments of the output data in the training dataset, in this case, the output binary masks, are computed from corner and middle points of the vertebrae manually labelled in the input data. The labelling algorithm uses the vertebral centers and the vertebral labels, which typically are C1-C7 for the cervical spine, T1-T10 for the thoracic spine and L1-L5 for the lumbar spine. More in detail, the vertebrae of a human spine are annotated and labelled in a semi-automatic way in the following process (see FIGS. 2a and 2b):
1. Identify and mark the middle of L1 and middle of T1 or top thoratic vertebra or the most cranial visible on image;
2. Identify the center line of spine and mark center of single vertebrae;
3. Check if a vertebra is out of the line, by checking if the distance is within the average distance of the vertebrae on top and below it and eventually manually correct it;
4. Draw polygons around the vertebrae for all vertebrae in a specific order so that the TopLeft TL is marked first, followed by TopRight TR, BottomLeft BL and BottomRight BR. In the case of a cervical certebra the TopMiddle TM and BottomMiddle BM points are also marked;
5. The vertebral centers are equal to the mean of the coordinates of the points belonging to the individual vertebral masks;

This annotation procedure results in a list of coordinates for all vertebrae from top (first cervical vertebra on image) to the bottom (last vertebra on image, usually L5) like T1, TopLeft TL (x,y), TopRight TR (x,y), etc. The list of annotated and labelled coordinates for all vertebrae is used as a basis for obtaining the segmentation masks. The output image segments are obtained by selecting a subset of coordinates based on the labels associated with the respective output mask and using the circumference of the selected subset of coordinates to obtain an image segment.

The segmentation image masks obtained from the annotated input medical images in the present example are the following:
An image mask 9 covering the entire spine (see FIG. 1d);
One image mask 10-13 for each spine segment (cervical 10, thoracic 11, lumbar 12, sacral 13; see FIG. 1e-h);
An image mask 14 covering the individual vertebrae (essentially, this is the same as the first image mask, but the intervertebral disks are not covered; see FIG. 1c).

For example, to obtain the image mask 9 covering the entire spine, the coordinates of all vertebrae are selected, the horizontal connections (between left and right coordinates) are omitted for all but the top and bottom vertebrae, and the adjacent top and bottom coordinates of neighboring vertebrae (based on the label) are connected. In this way, the perimeter/border/contour of the entire spine is obtained and then rendered into a binary mask by switching all pixels within the circumference to 1 and the remaining pixels outside the circumference to 0. For the spine segments, a similar procedure is performed, but limited to the coordinates of the vertebrae belonging to the respective segment, according to the associated vertebral label (C1-C7 for the cervical spine, T1-T12 for the thoracic spine and so forth). For the individual vertebrae segmentation mask, the perimeter/border/contour of each of the vertebrae is used to decide which pixels are switched to 1, leaving all remaining pixels at 0.

This finally results in a following annotated and labeled training data set: input 2D/3D spine images 1, 8, labelled list of vertebrae with coordinates TL, TR, etc. for corner points, wherein the labels are obtained with the algorithm described below, and various image masks 9-14 are derived therefrom.

For selecting the machine learning model architecture and devising the training procedure, the task to perform is semantic segmentation onto each of the output segmentations (i.e. image segments). Generally, for example any neural network able to perform the task can be used as the machine learning model. Moreover, it is also possible to decide whether to use a single machine learning model to learn all of the segmentation tasks or two machine learning models to separately learn vertebral segmentation and spine segment segmentation.

In the following we will proceed by assuming that two separate models 15, 16 (see FIG. 4) are used with an architecture 17 similar to the U-Net (see FIG. 3, which is FIG. 1 on the original U-net paper) proposed by O. Ronneberger, P. Fischer, and B. Brox, in their article “U-Net: Convolutional networks for biomedical image segmentation,” (Int'l. Conf. on Medical Image Computing & Computer-assisted Intervention (Springer, Cham, Switzerland, 2015), pp. 234-241). If not disclosed otherwise below, the parameters and encodings are used according to this article and are included herein by reference. Alternatively, it is possible to use a single machine learning model that has one output for each segmentation and is optimized using the sum of the vertebral segmentation losses and spine segmentation losses.

The U-Net architecture 17 is a fully convolutional network composed by a contracting part 18, using pooling layers 19 to decrease the input resolution 20, and an expansive path 21, using upsampling operators 22 instead of pooling layers. FIG. 3 illustrates the multi-channel feature maps as blue boxes, indicating the number of channels on top of the box and the x-y-size at the lower left edge of the box, and the different operations between the feature maps as arrows. Copied feature maps are represented as white boxes.

The convolutional layers can be initialized following the Xavier uniform initialization that draws samples from a uniform distribution within [−limit, limit], where limit=sqrt(6/(fan_in+fan_out)) (fan_in is the number of input units in the weight tensor and fan_out is the number of output units). An exemplary value for limit, in the case where fan_in =32 and fan_out=64, would be sqrt (6/(32+64))=0.25. More information is provided in the TensorFlow documentation, e.g. at https://www.tensorflow.org/api_docs/python/tf/keras/initializers/GlorotUniform.

Another option how the model parameters are initialized at the outset of the training can be that all model weights are random normally initialized. The optimizer is set to ADAM with learning rate 5.e-5. All other hyper parameters are set to Keras-standard parameters. More information is provided by the Keras documentation, e.g. at https://keras.io/api/optimizers/adam/, or by TensorFlow, e.g. at https://www.tensorflow.org/api_docs/python/tf/keras/optimizers/Adam.

The model layers are usually interleaved with (nonlinear) activation functions such as Sigmoid that do not need initialization (some functions may require setting an individual parameter, or the default setting is used). In order to optimize the training process it is possible to use data augmentation, that is random transformation of the input data spanning from simple rotation, translation, and scaling to more sophisticated elastic deformations (e.g. to simulate scoliosis). Finally, the models are optimized for segmentation tasks by minimizing the corresponding model losses using the backpropagation algorithm and any gradient based optimizer in a standard training loop after feeding the model with single images or batches of multiple images for several iterations until convergence of the corresponding loss.

FIG. 4 schematically illustrates the steps of segmenting anatomical structures in an original medical image 23 by using two machine learning models 15, 16 trained according to the present disclosure. The original medical image 23 is obtained of an X-ray. It is pre-processed as described above for the training input data to obtain the input data 24 comprising two medical images 25, 26. The first medical image 24 is a pre-processed version of the original medical image 23 after normalisation; the second medical image 25 is a contrast-enhanced version of the first medical image 24 after applying CLAHE. Both medical images 25, 26 are input to a first machine learning model 15 and to a second machine learning model 16. The first machine learning model 15 is trained for vertebrae semantic segmentation and has two output image segments 27, 28. The first output image segment 27 is an image mask of the entire spine. The second output image segment 28 is an image mask of the individual vertebrae. The second machine learning model 16 is trained for spine segment segmentation and has five output image segments 29-33. The first output image segment 29 again is an image mask of the entire spine. The second, third, fourth and fifth output image segments 30-33 are image masks of the cervical, thoracic, lumbar and sacral spine segments respectively.

The following sections will focus on the loss functions used to learn the tasks of vertebral and spine segment segmentation with the machine learning model architecture, initialisation and training data described above.

The task of vertebrae segmentation (i.e., that of the first machine learning model 15) consists of the prediction of an image segment 28 containing all the visible vertebrae. In order to overcome the limitation of previous approaches to vertebrae segmentation we propose to predict an additional image segment 27, the spine segmentation, containing the whole spine. Moreover, we use additional loss functions that, instead of being applied to one individual output, are applied to both output image segments in order to enforce the relationship between the segmented objects and improve the overall prediction. The final loss function that is optimized during model training is the following:

$$L = {L}_{spine} + {L}_{v ertebrae} + {L}_{aux}$$

It comprises three components, the first two, Lₛₚᵢₙₑ and L_{vertebrae} are standard segmentation losses applied to the predicted output and the target (ground truth) mask of the first image segment 27 and the second image segment 28, respectively. An example of such losses is the Dice loss, derived from the Dice coefficient as follows:

$${L}_{spine} = 1 - {D}_{spine}$$

$$D = \frac{2 {\sum }_{i}^{N} {p}_{i} {g}_{i}}{{\sum }_{i}^{N} {p}_{i}^{2} + {\sum }_{i}^{N} {g}_{i}^{2}}$$

where D is the Dice coefficient and the sums run over all the pixels N of the prediction p and the ground truth g. Other examples include pixel-wise binary cross entropy (BCE) or its combinations with the Dice loss over a single output mask.

The third component of the loss function Lis the auxiliary loss function Laux, which is defined as follows:

$${L}_{aux} = 1 - D ( \vert {p}_{spine} - {p}_{vertebrae} \vert , \vert {g}_{spine} - {g}_{vertebrae} \vert ) + W {\sum }_{i}^{N} ( 1 - {g}_{spine , i} ) {p}_{vertebrae , i}$$

This loss function is made of two main components. The first one is the Dice loss between the absolute values of the differences between the predicted spine mask (the first image segment 27) and vertebrae mask (the second image segment 28), and the corresponding ground truths; the second one is the sum of the intensities of the pixels in the predicted vertebrae mask that are not present in the spine mask ground truth, weighted by a factor W.

The first component focuses on the intervertebral disc spaces. The intervertebral disk space ground truth is obtained from the difference between the spine mask and the vertebrae mask. The absolute values are taken to force the corresponding differences to lay in the [0, 1] range that is the domain of the Dice coefficient. Minimizing this loss implicitly trains the model to learn about spaces between vertebrae and it is equivalent to computing a weighted Dice loss between the vertebrae prediction and ground truth where the error on pixels between vertebrae have double the weight. As a consequence, adopting this loss function will also lead to vertebrae masks where the vertebrae are separated better so that the risk of predicting aggregated vertebrae is reduced.

The second component focuses on the overlap between the predicted vertebrae and the area outside the spine ground truth. By minimizing this overlap, the model will learn that vertebrae can only be predicted inside the spine, which will minimize the risk of predicting false vertebrae in the wrong parts of the image. The weighting factor W can be used to normalize the measure of overlap to lay in the [0, 1] range like the other losses, as well as to give more or less importance to the overlap component of the auxiliary loss function.

Finally, further post-processing can be applied to the output masks to improve the segmentation quality (e.g. morphological operations or clustering algorithms to split aggregated vertebrae in the vertebrae mask).

In order to perform spine segment segmentation (i.e., the task of the second machine learning model 16), the machine learning model 16 needs to predict four segmentation masks (the output image segments 30-33), one for each spine segment: cervical, thoracic, lumbar and sacral (see also FIGS. 1e-h). Similarly to the case of vertebrae segmentation, we introduced another output for the whole spine (the first output image segment 29), as well as additional losses to model the dependencies between multiple outputs.

The loss function optimized for this task is the following:

$$L = {L}_{spine} + {L}_{c} + {L}_{t} + {L}_{l} + {L}_{s} + {L}_{aux}$$

As in the vertebrae case, the first five components of this loss are segmentation losses for individual outputs (e.g. Dice loss, BCE), while Laux is defined as follows:

$${L}_{aux} = 1 - D ( pmax ( {p}_{c} , {p}_{t} , {p}_{l} , {p}_{s} ) , {g}_{spine} ) + W {\sum }_{i}^{N} ( {p}_{c , i} + {p}_{t , i} + {p}_{l , i} + {p}_{s , i} - max ( {p}_{c , i} , {p}_{t , i} , {p}_{l , i} , {p}_{s , i} ) )$$

This auxiliary loss function is also made of two main components. The first component is the Dice loss between the pixel-wise maximum value between the individual spine segments predicted masks (or, generally, image segments), and the second one is the sum over all the positions N of the predictions of the difference between the sum of the individual pixel intensities and the corresponding maximum value.

In the first component, the pixel-wise maximum over the individual spine segments represents the combination of the spine segments in a single image mask. This mask is then compared with the ground truth of the spine mask using the Dice loss in order to force the model to learn that the sum of the individual spine segments should result in the whole spine.

The second component of Laux is a measure of the overlap between the individual segment masks. By minimizing this measure, the model learns that vertebrae or portions of the spine cannot be shared between segments. Therefore the algorithm will be able to assign the correct segment to every vertebra, improve the handling of ambiguous vertebrae, and finally perform vertebral labeling.

Vertebral labeling is illustrated in FIG. 5 with connections A and B to FIG. 4. It is performed after the segmentation tasks by using information from the obtained vertebral output image segment 28 and the spine segments output image segments 30-33. The algorithm works as follows:
1) Identify individual vertebrae in the vertebrae segmentation image segment 28 using a connected components algorithm (see Federico Bolelli, Stefano Allegretti, Lorenzo Baraldi, and Costantino Grana. Spaghetti Labeling: Directed Acyclic Graphs for Block-Based Connected Components Labeling. IEEE Transactions on Image Processing, 29(1):1999-2012, 2019. and Federico Bolelli, Stefano Allegretti, and Costantino Grana. One dag to rule them all. IEEE Transactions on Pattern Analysis and Machine Intelligence, 2021.)
2) Extract the centroid of each vertebra
3) Sort 34 the vertebrae in ascending order:
   a) Create a list of vertebrae, set the current index to 1
   b) Select the vertebra whose centroid has the lowest y coordinate and assign index 1, remove this vertebra from the list
   c) Select the closest vertebra to the vertebra at the current index from the vertebrae list
   d) Increase the current index and assign it to the selected vertebra
   e) Remove the vertebra from the list
   f) Go to point c until the vertebrae list is empty
4) Assign 35 vertebral type; for each vertebra
   a) Measure overlap between the individual vertebral segment and all spine segments according to the spine segment image segments 30-33
   b) Assign the type corresponding to the spine segment mask with the greatest overlap. If there is no overlap with any mask, don't assign any type; If the overlap is equal between multiple masks, assign the most caudal segment (e.g. it it is equally overlapping thoracic and lumbar maks, then assign thoracic)
5) Assign 36 vertebral labels:
   a) If there are any cervical vertebrae, select the lowest or most caudal cervical vertebra, assign decreasing labels starting from C7 and going up.
   b) If there are any thoracic vertebrae
      i) If there are cervical labels, select the highest or most cranial thoracic vertebra and assign increasing labels starting from T1 and going down
      ii) Else, if there are lumbar labels, select the lowest or most caudal thoracic vertebra and assign decreasing labels starting from the maximum between T12 and the number of thoracic vertebrae and going up (e.g. start from T12 if there are only 7 thoracic vertebrae, start from T13 if there are 13 thoracic vertebrae)
   c) If there are any lumbar vertebrae
      i) If there are thoracic labels, select the highest or most cranial lumbar vertebra and assign increasing labels starting from L1 and going down
      ii) Else, if there are sacral labels, select the lowest or most caudal lumbar vertebra and assign decreasing labels starting from the maximum between L5 and the number of lumbar vertebrae and going up (e.g. start from L5 if there are only 4 lumbar vertebrae, start from L6 if there are 6 lumbar vertebrae)
   d) If there are any sacral vertebrae, assign label S.

With the individual vertebrae segmented and their respective labels signed according to the above procedure, the corresponding image segments and labels can be overlaid over the original medical image 23 to obtain a segmented medical image 37. The segmented medical image 37 can be displayed to the medical practitioner.

1. A computer-implemented method for training a machine learning model,
wherein the machine learning model is configured to use a medical image as an input for generating an output comprising at least two image segments of the medical image,
wherein the training comprises optimizing a loss function measuring the predictive performance of the machine learning model for a set of training data to obtain a set of optimal model parameters of the machine learning model as result of the optimization,
characterized in that the loss function comprises a loss contribution that depends on a geometrical relationship between at least two of the image segments.

2. The method according to claim 1, characterized in that the machine learning model is configured to generate the at least two image segments as image masks, in particular binary masks, applicable to the medical image.

3. The method according to claim 1, characterized in that the training data comprises a plurality of medical training images and a corresponding plurality of image training segments, wherein each medical training image comprises a graphical representation of parts of an anatomy, wherein the corresponding image training segments indicate the anatomic parts represented in the respective medical training image.

4. The method according to claim 1, characterized in that the loss contribution depends on an overlap between at least two of the image segments.

5. The method according to claim 3, characterized in that the geometrical relationship represents a mereology relationship between at least two of the image segments, wherein from the at least two of the image segments a first image segment represents an anatomic whole and at least one second image segment represents an anatomic part of the anatomic whole.

6. The method according to claim 5, characterized in that the loss contribution depends on an exceedance of the first image segment by the at least one second image segment.

7. The method according to claim 5, characterized in that the loss contribution depends on the difference between the first image segment and the at least one second image segment.

8. The method according to claim 7, characterized in that the loss contribution is proportional to the size of the difference between the first image segment and the at least one second image segment.

9. The method according to claim 5, characterized in that the method involves at least two machine learning models, wherein each machine learning model is separately trained using a separate loss function that comprises a loss contribution that depends on a geometrical relationship representing a mereology relationship between at least two of the output image segments, wherein the machine learning models together generate an output comprising at least three image segments, wherein each machine learning model is trained on a different level of a hierarchy of mereology relationships between the output image segments.

10. The method according to claim 5, characterized in that from the image training segments corresponding to one medical training image a first image training segment represents an anatomic whole and at least one second image training segment represents an anatomic part of the anatomic whole, wherein the loss contribution further depends on the difference between a first image training segment and the at least one second image training segment.

11. A method for segmentation of anatomical structures in a medical image comprising the steps of:
training a machine learning model to obtain a set of optimal model parameters according to claim 1;
segmenting anatomical structures in said medical image by using said machine learning model with said optimal model parameters.

12. The method according to claim 11, wherein the medical image comprises a spine, a hand, a foot, a cell or an embryo.

13. A set of optimal model parameters for a machine learning model, wherein the machine learning model is configured to use a medical image as an input for generating an output comprising at least two image segments of the medical image, characterized in that the set of optimal model parameters has been obtained according to claim 1.

14. A computer program for segmentation of anatomical structures in a medical image, wherein the computer program comprises the set of optimal model parameters and the machine learning model according to claim 13 and instructions to cause a data processing apparatus to execute the step of segmenting anatomical structures in said medical image by using said machine learning model with said optimal model parameters.

15. A computer-readable medium having stored thereon the computer program of claim 14.
